(19) United States
(12) Patent Application Publication
(10) Pub. No.: US 2003/0212463 A1
(43) Pub. Date: Nov. 13, 2003

(54) Biocompatible and soft silicon penile prosthesis

(76) Inventor: Ju-Il Seo, Seoul (KR)

Correspondence Address: David J. Josephic, Wood, Herron & Evans, L.L.P., 2700 Carew Tower, 441 Vine Street, Cincinnati, OH, 45202-2917, US

(21) Appl. No.: 10/391,931

(22) Filed: Mar. 19, 2003

(30) Foreign Application Priority Data
Mar. 22, 2002 (KR) 2002-15668

(51) Int. Cl. A61F 2/26

(52) U.S. Cl. 623/23.72

(57) ABSTRACT

The present invention relates to a biocompatible and soft silicone penile prosthesis for enlarging a penis. More particularly, the present invention relates to a biocompatible and soft silicone penile prosthesis for enlarging a penis, the prosthesis containing silicon gel, elastomer, or physiological saline therein.

In addition, the present invention relates to a biocompatible and soft silicon penile prosthesis for enlarging a penis, the prosthesis containing silicon gel, elastomer, or physiological saline therein, being designed to be subcutaneously implanted into the penis above the corpus cavernosum of the penis, and having a C-like annular shape.

Furthermore, the present invention relates to a surgical implantation method for subcutaneously implanting said penile prosthesis into a penis above the corpus cavernosum of the penis.

### TECHNICAL FIELD

The present invention relates to a biocompatible and soft silicone penile prosthesis for enlarging a penis. More particularly, the present invention relates to a biocompatible and soft silicone penile prosthesis for enlarging a penis, the prosthesis containing silicon gel, elastomer, or physiological saline therein.

The present invention also relates to a method for surgically implanting the penile prosthesis of the present invention into the penis of a patient.

### BACKGROUND ART

There are numerous instances where males have surgery for enlarging their penises in order to resolve their sexual frustration or the malfunction of their penises which in many cases results from the small size of their penises and thereby from the lack of psychological well-being about their sexual activity. Such a surgery for enlarging penises includes fat grafting, skin grafting, solid silicon prosthesis insertion etc.

Skin grafting is rather a complex surgical operation for enlarging a penis in which skin tissues of a male are extracted from an abdominal or inguinal region and then subcutaneously implanted into the penis of the same male. The drawbacks of such an operation are as follows: (i) a big scar remains after the surgery; and (ii) new blood vessels do not appropriately generate inside implanted skin grafts, which in turn tend to undergo necrosis.

Fat grafting is rather a simple surgical operation for enlarging a penis in which fat tissues of a male are extracted from an abdomen or buttock and then subcutaneously implanted into the penis of the same male. However, over the time after the surgical operation, a problem occurs that the implanted fat tissues tend to be absorbed into the subcutaneous tissues of the penis.

Solid silicon prosthesis insertion, which has been widely performed, is rather an expensive and complicated surgical operation for enlarging a penis in which a solid silicon prosthesis is subcutaneously implanted into the penis. However, in many cases, due to the solid silicon prosthesis, capsules and stiffness are generated within a penis after implantation, causing pain in both the male and female during their sexual interaction. Also, the girth of a penis generally increases only by about 1 cm, thus rendering such a prosthesis insertion ineffective in enlarging a penis. Furthermore, after said prosthesis is implanted, the penis does not look natural and has even an ugly appearance in many cases.

Aside from the above described surgical methods, to be even worse, some unqualified persons have been performing surgical operations for enlarging a penis in which a variety of materials unsuitable and unapproved for implanting into a penis (such as, paraffins, vaselines, plastics, metals, solid rings etc.) are implanted into the penis, thereby causing severe complications like inflammations, disease infections, skin necrosis, chronic pain etc.

Therefore, there is a need to develop a penile prosthesis for enlarging a penis, which is made of soft and biocompatible materials similar to human tissues and thus causes no feeling of foreign materials inside the penis; can be kept permanently inside the penis after implantation without any adverse side effect (inflammations, pain etc.); is not absorbed into the neighboring subcutaneous tissues of the penis over the time after implantation; and thus can reduce the psychological complex about sexual activity some males have due to the small size of their penises.

In this regard, until now, soft silicon products such as silicon gel have been widely employed for breast enlargement in females, not for penis enlargement in males.

### SUMMARY OF THE INVENTION

The present invention provides a biocompatible and soft silicone penile prosthesis for enlarging a penis, the prosthesis containing silicon gel, elastomer, or physiological saline therein.

In a preferred embodiment, the prosthesis of the present invention is designed to be subcutaneously implanted into a penis above the corpus cavernosum of the penis.

The present invention also provides a biocompatible and soft silicone penile prosthesis for enlarging a penis, wherein the prosthesis contains silicon gel or physiological saline; is designed to be subcutaneously implanted into the penis above the corpus cavernosum of the penis; and has a C-like annular shape.

The width of the prosthesis is preferably about 2 to about 4 cm, and the thickness of the implant is preferably about 0.3 to about 0.7 cm.

In another preferred embodiment, the thickness of the prosthesis gradually decreases from the center along to the ends of the prosthesis, and the ends of the prosthesis are substantially arch-shaped and gently curved.

In another embodiment, the prosthesis further has on its outward surface a well-sealed passage for injecting thereinto and/or taking out therefrom said silicon gel, elastomer, or physiological saline.

The present invention also provides a surgical implantation method for implanting a penile prosthesis according to the present invention into a penis of a patient, which comprises incising, above the pubic bone area of said patient, a sufficient length of the abdominal region of said patient; and subcutaneously implanting said implant into said penis above the corpus cavernosum of said penis.

In a preferred embodiment, the sufficient incision length of said method is about 3-5 cm.

### BRIEF DESCRIPTION OF THE ACCOMPANYING DRAWINGS

Preferred embodiments of the invention are illustrated by the accompanying drawings, in which:

FIG. 1 is a schematic side elevation view of the penile prosthesis (1) according to the preferred embodiment of the present invention, which has on its outward surface a well-sealed passage (2) for injecting thereinto and/or taking out therefrom silicon gel, elastomer, or physiological saline.

FIG. 2 is a schematic cross-section view of a state where the penile prosthesis (1) according to the preferred embodiment of the present invention is implanted into between the cutaneous area (3) and the corpus cavernosum (4) of a penis (S).

FIG. 3 is a schematic longitudinal section view of FIG. 2, in which W and D are the width and thickness of the penile prosthesis, respectively.

### DETAILED DESCRIPTION OF PREFERED EMBODIMENTS

The present invention relates to a biocompatible and soft silicone penile prosthesis for enlarging a penis, wherein the prosthesis contains silicon gel, elastomer, or physiological saline.

Preferably, the penile prosthesis according to the present invention is designed to be subcutaneously implanted into a penis above the corpus cavernosum of the penis (FIG. 2).

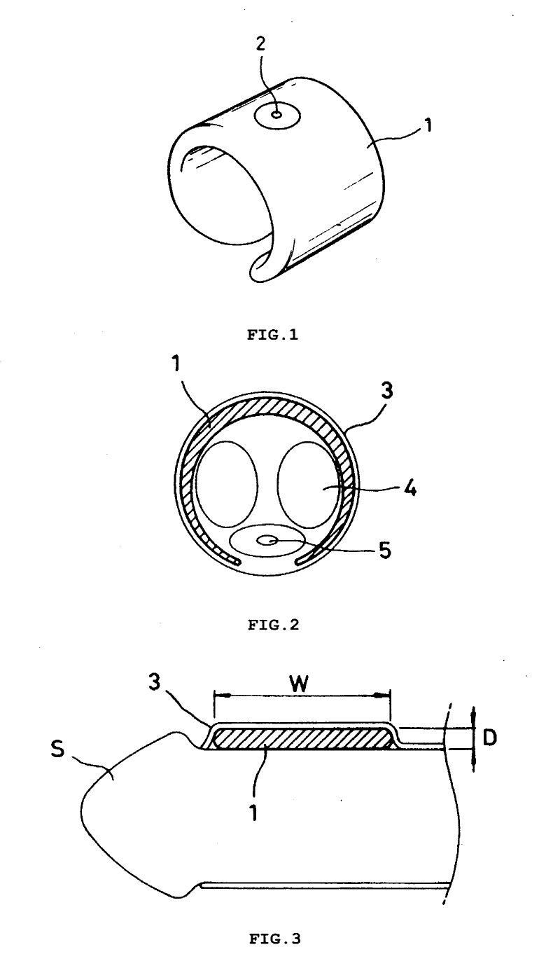

The present invention also relates to a biocompatible and soft silicone penile prosthesis for enlarging a penis, wherein the prosthesis contains silicon gel or physiological saline; is designed to be subcutaneously implanted into the penis above the corpus cavernosum of the penis; and has a C-like annular shape (FIGS. 1 and 2).

Preferably, the width and thickness of said penile prosthesis is about 2-4 cm and about 0.3-0.7 cm, respectively.

Furthermore, in another embodiment of the present invention, the thickness of the penile prosthesis gradually decreases from the center along to the ends of the prosthesis, and the ends of the prosthesis are substantially arch-shaped and gently curved (FIGS. 1 and 2).

Instead of hard or stiff materials (e.g. solid silicon), the penile prosthesis according to the present invention is made of a “biocompatible and soft silicon” material which does not cause an immune response when implanted and delivers a feeling of comfort or of substantially having no foreign materials to a male having the penile prosthesis implanted.

For the purpose of the present invention, any biocompatible and soft silicon products can be used which silicon gel, elastomer, or physiological saline is already contained therein or which silicon gel, elastomer, or physiological saline is to be injected thereinto and/or taken out therefrom when necessary (e.g. right before when the penile prosthesis of the present invention is implanted). More preferably, silicon gel-containing soft silicon products are used which are being widely employed in a surgical implantation for breast enlargement in females (for example, silicon gel products from DOW CORNING Corporation, US).

In a preferred embodiment where silicon gel or physiological saline is to be injected into and/or taken out from the penile prosthesis of the present invention when necessary, the penile prosthesis further has on its outward surface a well-sealed passage for injecting thereinto and/or taking out therefrom said silicon gel, elastomer, or physiological saline. More preferably, said means is located on around the center region of its outward surface. Such a means is used for injecting thereinto and/or taking out therefrom said silicon gel, elastomer, or physiological saline to adjust the girth or thickness of the prosthesis when necessary. When adjusting the girth or thickness of the prosthesis, conventional injectors etc. can be used for injecting thereinto and/or taking out therefrom said silicon gel, elastomer, or physiological saline.

The term “outward surface” of the penile prosthesis means the surface of the prosthesis which is oriented toward the cutaneous region of a penis, not toward the corpus cavernosum of a penis (FIG. 2).

FIG. 1 shows a preferred embodiment of the biocompatible and soft penile prosthesis (1) having C-like annular shape. More specifically, the penile prosthesis of FIG. 1 contains silicon gel therein, and its thickness gradually decreases from the center (about 0.5 cm) along to the ends thereof (about 0.3 cm). In addition, both ends of the prosthesis are substantially arch-shaped and gently curved.

Also, the penile prosthesis as shown in FIG. 1 further has on around the center region of its outward surface a well-sealed passage (2) for injecting thereinto and/or taking out therefrom said silicon gel.

The present invention also includes a surgical implantation method by which the above described penile prosthesis is implanted into the penis of a patient, which comprises

incising, above the pubic bone area of said patient, a sufficient length of the abdominal region of said patient; and

subcutaneously implanting said prosthesis into said penis above the corpus cavernosum of said penis.

The term “the abdominal region above the pubic bone area of said patient” (not shown) means an abdominal area located about 1 cm from the base of a penis along the abdomen of a patient.

The sufficient incision length in said method depends primarily on the width of the prosthesis. Preferably, the sufficient incision length in said method is about 3-5 cm.

In this regard, FIGS. 2 and 3 respectively show a schematic cross-section and longitudinal section view of a state where the penile prosthesis according to said preferred embodiment of the present invention is implanted into between the cutaneous area (3) and the corpus cavernosum (4) of a penis (S).

As shown in FIGS. 1 and 2, when implanted, the C-like annular shape of said penile prosthesis can sufficiently encircle the corpus cavernosum (4) and the spongy penile urethra (5) of a penis (S).

The penile prosthesis according to the present invention has following advantages:

(i) it can be kept permanently inside a penis after implantation without any adverse side effect;

(ii) it is not absorbed into the neighboring subcutaneous tissues of the penis over the time after implantation, unlike fat grafting;

(iii) it is not degenerated or decomposed over the time after implantation;

(iv) no capsules and stiffness are generated within the penis after implantation, resulting in no feeling of foreign materials inside the penis and in no pain;

(v) a surgical implantation method according to the present invention per se is rather easy to implement, and scar after the surgical implantation is rather small; and

(vi) it is very effective for enlarging a penis by increasing the size of the penis which amounts to its thickness of about 0.3-0.7 cm and width of about 2-4 cm.

While the present invention has been described primarily with reference to said preferred embodiments and accompanying Figures, it will be clearly understood by a person skilled in the art that the present invention is not limited thereto. Rather, it will be obvious to a person skilled in the art that many modifications, variations and equivalents of the above described prostheses are possible without departing from the inventive concept of the present invention. Such modifications, variations and equivalents thus lie within the spirit and scope of the present invention as defined in the appended claims.

What is claimed is:

1. A biocompatible and soft silicone penile prosthesis for enlarging a penis, wherein the prosthesis contains silicon gel, elastomer, or physiological saline.

2. The prosthesis as claimed in claim 1, wherein the prosthesis is designed to be subcutaneously implanted into a penis above the corpus cavernosum of the penis.

3. A biocompatible and soft silicone penile prosthesis for enlarging a penis, wherein the prosthesis contains silicon gel, elastomer, or physiological saline therein; is designed to be subcutaneously implanted into the penis above the corpus cavernosum of the penis; and has a C-like annular shape.

4. The prosthesis as claimed in claim 3, wherein the width of the prosthesis is about 2 to about 4 cm; and the thickness of the prosthesis is about 0.3 to about 0.7 cm.

5. The prosthesis as claimed in claim 3 or 4, wherein the thickness of the prosthesis gradually decreases from the center along to the ends of the prosthesis; and the ends of the prosthesis are substantially arch-shaped and gently curved.

6. The prosthesis as claimed in claim 3 or 4, wherein the prosthesis further has on outward surface thereof a well-sealed passage for injecting thereinto and/or taking out therefrom said silicon gel, elastomer, or physiological saline.

7. A surgical implantation method for implanting a penile prosthesis as claimed in any of claims 1 to 6 into a penis of a patient, which comprises
incising, above the pubic bone area of said patient, a sufficient length of the abdominal region of said patient; and
subcutaneously implanting said prosthesis into said penis above the corpus cavernosum of said penis.

8. The surgical implantation method as claimed in claim 7, the sufficient incision length is about 3-5 cm.
